(19) United States
(12) Patent Application Publication
(10) Pub. No.: US 2024/0350691 A1
(43) Pub. Date: Oct. 24, 2024

(54) CONTINUOUS FLOW, HIGH THROUGHPUT APPARATUS AND METHOD FOR INACTIVATING VIRUSES AND PATHOGENS IN HUMAN PLASMA

(71) Applicant: Trevor Percival Castor, Arlington, MA (US)

(72) Inventor: Trevor Percival Castor, Arlington, MA (US)

(73) Assignee: Aphios Corporation, Woburn, MA (US)

(21) Appl. No.: 18/031,574

(22) PCT Filed: Oct. 12, 2021
(86) PCT No.: PCT/US21/54613

§ 371 Date: Apr. 12, 2023

(51) Int. Cl. A61L 2/24; A61L 2/00

(52) U.S. Cl. CPC A61L 2/24; A61L 2/0011; A61L 2202/14; A61L 2202/15; A61L 2202/16; A61L 2202/22

(57) ABSTRACT

The present invention is for a continuous-flow pathogen reduction apparatus and method, based purely on pathogen inactivation physical principles, for controlling or eliminating trans fusion-transmittable infections from emerging pathogens, pandemic viruses, and bioterrorism threats. The invention inactivates both nonenveloped and enveloped viruses as well as pathogenic bacteria and parasites in human plasma and biologies, while retaining the natural bioactivity, integrity and potency of the treated biologic. The method uses critic al, near-critical or supercritical fluids for viral and pathogen reduction of plasma and biologies. The apparatus is designed to rapidly process high volumes of plasma and biologies with high levels of pathogen reduction in a continuous flow fashion.

### FIELD OF INVENTION

The present invention is directed to methods and apparatus for inactivating enveloped and nonenveloped viruses and other pathogens in units of whole blood to prevent transfusion-transmitted infections. The invention features a high flow rate plasma processing apparatus using critical, supercritical, or near critical fluids for inactivation of viruses and pathogens.

### REFERENCES TO OTHER PATENTS

This application discloses a number of improvements and enhancements to the viral inactivation method and apparatus disclosed in U.S. Pat. No. 5,877,005 to Castor et al., which is hereby incorporated by reference in its entirety.

This application discloses a number of improvements and enhancements to viral inactivation method and apparatus disclosed in U.S. Pat. No. 6,465,168 to Castor et al., which is hereby incorporated by reference in its entirety.

This application discloses a number of improvements and enhancements to the method for inactivating viruses for use in vaccines as disclosed in U.S. Pat. No. 7,033,813 to Castor et al., which is hereby incorporated by reference in its entirety.

This application discloses a number of improvements and enhancements to the method for inactivating viruses as disclosed in published U.S. Patent Application No. 2006/0269928 to Castor, which is hereby incorporated by reference in its entirety.

This application is being filed simultaneously on the same date with related inventions as disclosed in U.S. Provisional Patent Applications Nos. 63/090,707, 63/090,711 and 63/090,713 to Castor, which are hereby incorporated by reference in their entirety.

### BACKGROUND OF THE INVENTION

Viruses of many types pose an increasing and serious worldwide threat to humans and animals. The recent emergence of SARS-CoV-2, the etiologic agent of our ongoing COVID-19 pandemic that has taken almost 5 million lives around the world and over 700,000 in the United States, has caused catastrophic damage to human health and safety, and untold economic damage to both the developed but especially the developing world. The rapid spread of the Zika virus can have a significant impact on neurological disorders in unborn fetuses and potentially adults. Outbreaks of the extremely virulent Ebola virus, the periodic emergence of severe viral respiratory infections (e.g. SARS), recurrent outbreaks of potentially pandemic strains of influenza (e.g. H5N1), and the worldwide AIDS epidemic have highlighted the need for effective methods and devices for the inactivation and removal of pathogens from human blood plasma and plasma-derived products.

Emerging viruses like West Nile, the Mexican swine flu, and high-risk bioterrorism pathogens such as smallpox are all major threats to the safety of the human plasma supply chain. In addition to viruses, bacteria and parasites such as Babesia spp. and Plasmnodiwn spp. can cause severe transfusion-transmitted infectious diseases. The causes of the more rapid emergence and spread of these “killer” viruses and pathogens are not entirely known, but they are thought to be caused by some combination of deforestation with urbanization of wild virus habitats, evolutionary mutations and rapid global travel.

Even with the increased emphasis on screening of donors and testing for pathogens, the risk of transmission of infection by blood products is currently estimated to be 1 in 205,000 for hepatitis B virus (HBV), 1 in 2 million for hepatitis C virus (HCV), and 1 in 2 million for HIV (NHLBI, NIH, 2016). These numbers are significantly higher when disaggregated by region or socio-demographic clusters within and outside of the US. In addition, viruses such as human T-cell lymphotropic viruses (HTLV) types I and II, human parvovirus B19, cytomegalovirus (CMV) and Epstein-Barr virus (EBV) pose potential risks following the transfusion of blood and blood components.

Viruses of major concern as pathogens in human blood plasma include such as human parvovirus B19 and the hepatitis A, B and C viruses (non-enveloped viruses), and the enveloped viruses like human immunodeficiency viruses HIV-1 and HIV-2, and herpes viruses (CMV, EBV, HHV-6, HHV-7, HHV-8). For most of these viruses, the short period between initial infection and seroconversion presents a window of significant transfusion risk as routine clinical tests are not sufficiently robust to detect the virus. CMV seroprevalence, for example, may range from 40%-100% depending on locale, and establishes as a life-long latent infection with severe morbidity to patients.

Annually, an estimated 3.8 million Americans are transfused with 28.2 million blood components derived from 12.8 million units of blood donated by apparently healthy volunteers. A rigorous scrutiny of blood donors and screening of donated blood for various serological markers have significantly reduced the mortality and morbidity due to transfusion-transmitted infectious diseases; however, some enzyme immunoassays used for routine screening may detect viral antigens or antibodies, but not the infectious agents themselves.

Moreover, reliance on patient disclosure and risk assessment questionnaires has previously failed to correctly identify virus infected blood donors (CDC, 2010). Thus, there could be an asymptomatic window period of infectivity responsible for a residual risk of post-transfusion infection. Whereas conservative data estimates 1 in every 1.5 million are at a risk of transfusion transmitted infection (TTI) in the U.S (CDC, 2010), increasing globalization of public health increases the risk of TTI to Americans substantially.

A number of emerging viruses such as SARS Coronavirus, Zika, West Nile, the Mexican swine flu, and other potential bioterrorism pathogens like smallpox are not conventionally screened for, but are of concern to the safety of the human plasma supply chain. Additionally, microorganisms like bacteria, Babesia spp and Lyme, and parasites like malaria are also not conventionally screened for, but are a major threat of spreading diseases through transfusions.

Moreover, the high frequency episodes of emergent and seasonal infectious diseases like Ebola, Zika, MERS, SARS, West Nile virus (WNV), and rare yet fatal blood borne infectious agents like Prion Diseases and Variant Creutzfeldt-Jakob Disease (vCJD), continue to cause localized and global-level public health risk from TTI. Combined, these risks supersede the risks reported within individual country borders (CDC, 2013). In many developing countries, the risk of being transfused with donor blood infected with at least one pathogen (including HIV, HCV & HBV) is substantially high, ranging from 9.5%-21.1% in Ethiopia to 1.2%-15% in China. These risks and those of emergent infectious epidemics like Zika, follow regional and socio-demographic trends, from which developed countries cannot be precluded.

New products and processes that address the variable but important global burden of transfusion transmitted infection (TTI) wvill provide safer blood products that will benefit public health and also provide significant commercial innovations.

Several pathogen inactivation technologies have been used, but they have drawbacks. Current approaches such as pasteurization, solvent-detergent (SD), UV irradiation, and chemical and photochemical inactivation are not always effective against a wide spectrum of pathogens. Known approaches are sometimes encumbered by process-specific deficiencies, and often result in denaturation of the biologics that they are designed to render safe. There are limited commercially available, FDA-approved technologies for the inactivation of non-enveloped viruses, which could pose a significant future threat to the safety of human plasma and biologics. Currently, there are no drugs effective against multiple viral agents.

There is an immediate need for a technology to inactivate viruses and other pathogens from units of human plasma, particularly in developing countries and hot zones, while maintaining the biologic integrity of the plasma. An effective technology would be capable of processing high flow rates of blood plasma to accommodate the rapidly expanding need to remove pathogens from high volumes of blood plasma, particularly in the case of spreading diseases, pandemics, and bioterrorism threats, which are more common in the world, and where the integrity and potency of blood supplies must be ensured.

### SUMMARY OF THE INVENTION

The present invention is a generally applicable technology, based on physical principles, for the inactivation of both enveloped and nonenveloped viruses in units of human plasma with minimal reduction in biological integrity and potency. This technology reduces the risk of transfusion-mediated transmission of known as well as unknown pathogens and potential bioterrorism threats. The present invention is capable of processing blood plasma in high flow rates on a daily basis to control transfusion-borne transmission of viruses and other pathogens.

The drawbacks of prior known approaches are remedied by the present invention. In one aspect, the present invention is a physical pathogen inactivation technology and apparatus, in the form of a multistage system which can process from 10 liters to 1000 liters of human plasma per day, for the inactivation of both non-enveloped and enveloped viruses as well as pathogenic bacteria and parasites in units of human plasma. Apparatus of the present invention is up-scalable to process higher flow rates of blood plasma as necessary to ensure the integrity of the blood supply.

In another aspect of this invention, this technology utilizes supercritical and near-critical fluids (SuperFluids™ or SFS). SFS are normally gases at ambient conditions of temperature and pressure, which when compressed, exhibit enhanced thermodynamic properties of solvation, penetration, selection and expansion. These gases are used to permeate and inflate virus and pathogen particles. When the pressure in the SFS-saturated particles is released, the particles rupture at their weakest points as a result of rapid phase conversion and the forces of expansion. The present inventor has demonstrated that the CFI™ (critical fluid inactivation) process inactivates both enveloped viruses such as MuLV, VSV, Sindbis, HIV (all completely inactivated), TGE, and BDVD, and the nonenveloped viruses Polio, Adeno, EMC (complete), Reo, and Parvo, while preserving biological activity of the CFI-treated product. The aspect of preserving the protein integrity and biological activity of the CFI-treated product is a major advantage over prior technologies and approaches.

Conventional approaches for pathogen inactivation in biologics are not always effective against a wide spectrum of human and animal viruses, and are sometimes encumbered by process-specific deficiencies, and often result in denaturation of the biologicals that they are designed to protect. CFI pathogen inactivation technology gives pathogens the “bends,” inactivating them without damaging proteins and enzymes in medically important transfusion fluids such as human plasma.

In research collaboration with the National Institute of Biological Standards and Control (NIBSC), London, England, the inventor demonstrated that CFI technology inactivated more than 4 logs of human Parvovirus B19 (one of the smallest and most resilient viruses) in human plasma in a two-stage CFI unit in less than 20 seconds. The inventor also demonstrated that SFS can disrupt and inactivate microorganisms such as E. coli, thick-walled prokaryotes such as Bacillus subtilis, and tough eukaryotes such as Saccharonyces cerevisiae at same SFS conditions for inactivating viruses. At the inventor's company, Aphios Corporation, scientists and engineers have defined operating conditions for achieving >6 logs of virus inactivation of prototypical enveloped and non-enveloped viruses in pooled human plasma in a 2-stage laminar flow CFI unit with retention of >80% of protein integrity.

The effect of treatment with different ratios of SFS CO₂ and N₂O on pH and coagulation factors in human plasma was evaluated in a two-stage CFI unit used for optimizing SFS composition. A plasma optimized SFS mixture consisting of N₂O:CO₂::97.5:2.5 inactivated 3.4 logs of the enveloped BVDV in pooled human plasma in the two-stage CFI unit at 208 bars and 37° C., and 4.1 logs of the non-enveloped Adenovirus Type 2 in FBS at 208 bars and 40° C. in a recycling, single-stage CFI unit.

The present invention, based on CFI technology, is a purely physical technique that does not involve the use of heat, chemicals and/or irradiation, each of which has significant drawbacks in the viral inactivation of human plasma. As such, while CFI is capable of inactivating wide classes of viruses, bacteria and parasites, it has negligible negative impact on biological integrity and potency of the treated fluids. The potential impact of a generally applicable, physical technology for inactivating viruses and emerging pathogens with high retention of biological activity is highly significant. CFI technology will also be vital for developing countries and hot zones for the clearance of viruses from human plasma.

These and other features, aspects and advantages of the present teachings will be better understood with reference to the following drawings, description, examples, and appended claims.

### BRIEF DESCRIPTION OF THE DRAWINGS

FIG. 1 shows before-and-after TEM (Transmission Electron Microscopy) photomicrographs of normal viral activity before CFI and after CFI disruption and inactivation of bacteriophage Φ-virus;

FIG. 2 shows before-and-after SEM (Scanning Electron Microscopy) photomicrographs of normal viral activity before CFI and after CFI disruption and inactivation of yeast (Saccharonyces cerevisiae);

FIG. 3 schematically illustrates (a) single-stage and (b) multiple-stage SuperFluids™ viral inactivation devices;

FIG. 4 shows inactivation of HIV-1 by Different SuperFluids™ at 3,000 psig and 22° C.

Virus-containing supernatant was diluted 1:10 in RPMI and run through the CFI-unit with different SuperFluids™ conditions. HIV-1_{Δtat-rev} was used for each run. An aliquot was not exposed to SuperFluids™ and served as a time and temperature control. 10-fold serial dilutions of the control and treated samples were made and used in the TCID₅₀ assay to measure infectious virus. It was noted that cells at the top dilution of virus (1:10) did not grow, and therefore were not included when calculating the TCID₅₀. Thus, the limit of detection for this assay is 2.7 logs. N₂O/CO₂—N₂O with trace quantities of CO₂; N₂O+5% CO₂— a mixture of 95% N₂O and 5% CO₂ by volume; n Control; n CFI-Treated;

FIG. 5 is a graph showing Proteostat aggregation assay results for CFI-075 and CFI-076;

FIG. 6 is a graph showing the effect of CFI N₂O at 152 bars and 22° C. on Fibrinogen;

FIG. 7 is a schematic showing a two-stage process SFS-CFI unit;

FIG. 8 is a schematic illustration of the plasma stream recycle concept of the present invention;

FIG. 9 is a schematic illustration of a commercial scale SFS-CFI prototype for processing to 1,000 Liters of blood plasma per day of the CFI bench-top unit process flow diagram;

FIG. 10 a schematic illustrating the process flow of plasma stream recycle CFI module as illustrated in FIG. 7;

FIG. 11 illustrates the process flow of a three-stage CFI module; and

FIG. 12 illustrates the process flow of a five-stage CFI module.

### DETAILED DESCRIPTION OF THE PREFERRED EMBODIMENTS

Viruses of all types pose an increasing serious worldwide threat. The rapid spread of the Zika virus, which can have a significant impact on neurological disorders in unborn fetuses and potentially adults, the recent outbreak of the extremely virulent Ebola virus, periodic emergence of SARS, recurrent outbreaks of potentially pandemic strains of influenza such as H5N1, the continuing epidemic of MERS and the worldwide AIDS epidemic have highlighted a persistent concern in the health-care community—the need for effective pathogen inactivation and removal techniques for human blood plasma and plasma-derived products.

CFI™ (Critical Fluid Inactivation) utilizes supercritical and near-critical fluids (SuperFluids™ or SFS). SuperFluids™ are normally gases which, when compressed, exhibit enhanced thermodynamic properties of solvation, penetration, selection and expansion. These gases are used to permeate and saturate virus and pathogen particles. The SFS-saturated particles then undergo decompression and, as a result of rapid phase conversion, viruses inflate and rupture at their weakest points.

The present inventor has demonstrated that the CFI™ (critical fluid inactivation) process inactivates both enveloped viruses such as MuLV, VSV, Sindbis, HIV (all completely inactivated), TGE, and BDVD, and the non-enveloped viruses Polio, Adeno, EMC (complete inactivation), Reo, and Parvo viruses, while preserving biological activity of the CFI-treated product. In research collaboration with the National Institute of Biological Standards and Control (NIBSC), London, England, the CFI process inactivated more than 4 logs of human Parvovirus B19 (one of the smallest and toughest viruses) in human plasma in a two-stage CFI™ unit in less than 20 seconds.

It has also been demonstrated that SFS can disrupt and inactivate microorganisms such as E. coli, thick-walled prokaryotes such as Bacillus subtilis and tough eukaryotes such as Saccharonyces cerevisiae at viral inactivation SFS conditions. CFI can be used with viral reduction methods such as nanofiltration as an orthogonal method of pathogen clearance, and is versatile for refinement to treat cellular blood. The present data have been generated using prototypes of a pilot-scale CFI unit.

This invention can be used is to construct high-flow rate CFI units for blood banks, and through licensing agreements, provide equipment and technology transfer as well as prevention and maintenance support to blood banks. This invention is for a generally applicable technology, based on physical principles, for the inactivation of both enveloped and non-enveloped viruses in units of human plasma with minimal reduction in biological integrity and potency in order to reduce the risk of transfusion-mediated transmission of known as well as unknown pathogens and potential bioterrorism threats.

The present invention is a physical pathogen inactivation technology, or Critical Fluid Inactivation (CFI™), for the inactivation of both non-enveloped and enveloped viruses as well as pathogenic bacteria and parasites in human plasma, plasma protein products and biologics. CFI™ technology is applicable to both units of plasma and pooled human plasma, the more globally significant focus of the current application.

Currently, there is no commercially available, FDA-approved technology for the inactivation of nonenveloped viruses in units of pooled human plasma and biologics, and only one approved method for units of plasma, which can inactivate some, but not all known non-enveloped viruses. This dearth of FDA-approved pathogen inactivation technologies poses a significant future threat for known and new viruses in human plasma and biologics.

A number of approaches have been employed for the inactivation or removal of viruses in human plasma, harnessing therapeutic proteins derived from human plasma and preparation of recombinant biologics. These include heating or pasteurization; solvent-detergent technique; Ultraviolet (UV) irradiation; chemical inactivation utilizing hydrolysable compounds such as β-propiolactone and ozone; and photochemical decontamination using synthetic psoralens. The major problems with pasteurization include long pasteurization times, deactivation of plasma proteins and biologics, and the use of high concentrations of stabilizers that must be removed before therapeutic use. The solvent-detergent (SD) technique is quite effective against lipid-coated or enveloped viruses such as HIV, HBV and HCV, but is ineffective against protein-encased or non-enveloped viruses such as HAV and parvovirus B19. The solvent-detergent technique is also burdened by the need to remove residual organic solvents and detergents before therapeutic use. The photochemical-psoralen method, while quite effective with a wide range of viruses, is burdened by potential residual toxicity of photoreactive dyes and other potentially carcinogenic or teratogenic compounds.

However, the Cerus Intercept method that is effective against both enveloped and some but not all non-enveloped viruses has been recently approved by the FDA for the viral clearance of human plasma, red blood cells and platelets. HAV, HEV, B19, and Polio Virus are resistant to the Cerus inactivation process, but are sensitive to the present CFI technology. Moreover, the Intercept method is restricted to units of plasma and is not applicable to pools of plasma, an advantage that the CFI offers since it was initially developed for pools of human plasma. The major weakness of CFI is that it has not yet been optimized for cellular blood e.g. platelets, an advantage Cerus' Intercept offers. However, CFI offers superiority in breadth in the number, types and strains of pathogens completely inactivated, with an accompanying simplicity, versatility and cost-efficiency. Thus, current approaches are not always effective against a wide spectrum of human and animal viruses, are sometimes encumbered by process-specific deficiencies, and often result in denaturation of the target biologics.

CFI technology, which inactivates both enveloped and non-enveloped viruses, is applicable to both pooled human plasma and units of plasma. The potential impact of a generally applicable, physical technology for inactivating both enveloped and non-enveloped viruses and emerging pathogens with high retention of biological activity is thus very significant. Such a technology, especially when used with conventional virus inactivation or removal methods such as nanofiltration, will help ensure a blood supply that is safe from emerging and unknown pathogens and bioterrorism threats. In addition to human plasma and human plasma proteins such as fibrinogen and immunoglobulins, the developed technology will also be applicable to monoclonal antibodies and transgenic molecules.

The technology could be very impactful in developed countries and in hot zones for both the rapid virus clearance of pooled human plasma and units of plasma. The inventor developed two prototypes of this technology with versatility and cost efficiency that include; (i) an inexpensive bench-top prototype device that uses customized blood bags and can be readily deployed at community-level points-of-need where outbreaks occur, and (ii) pilot and large scale CFI units to maximize high throughput processing at blood banks and hospitals, and industries (Industrial prototype). Both prototypes operate under similar CFI process conditions and use similar principles for pathogen inactivation. The technology offers unique advantages not achievable by currently available competing products like that of SD and the Cerus Intercept.

CFI™ pathogen inactivation works, in part, by first permeating and inflating the virus particles with a selected Superfluid™ under pressure. The overfilled particles are then quickly decompressed, and the dense-phase fluid rapidly changes into gaseous state rupturing the virus particles at their weakest points—very much like the embolic disruption of the ear drums of a scuba diver who surfaces too rapidly. The disruption of viral structure and release of nucleic acids prevents replication and infectivity of the CFI treated viral particle.

SuperFluids™ (SFS) of interest are normally gases, such as carbon dioxide and nitrous oxide, at room temperature and pressure. When compressed, these gases become dense-phase fluids, which have enhanced thermodynamic properties of selection, solvation, penetration and expansion. The ultra-low interfacial tension of SuperFluids™ allows facile penetration into nanoporous and microporous structures. As such, SFS can readily penetrate and inflate viral particles. Upon decompression, because of rapid phase conversion, the overfilled particles are ruptured and inactivated (Castor et al., 1995, 1999, 2000, 2001, 2002, 2005, 2006).

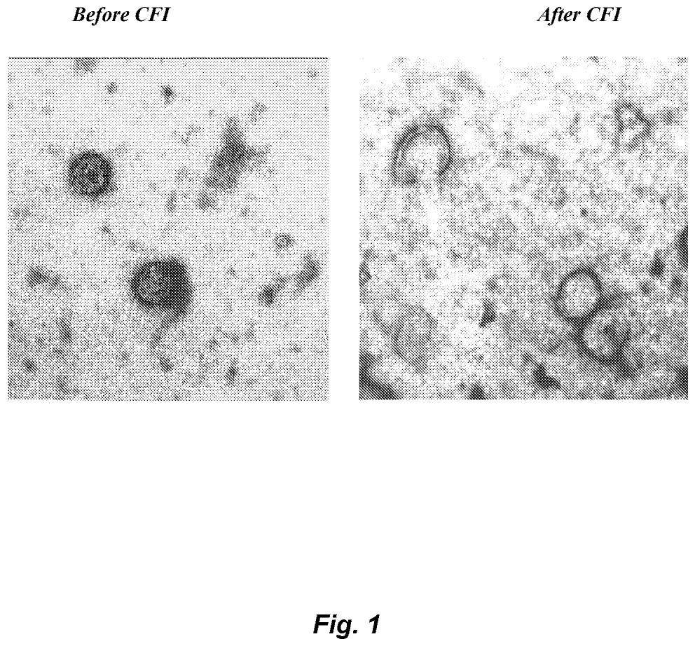

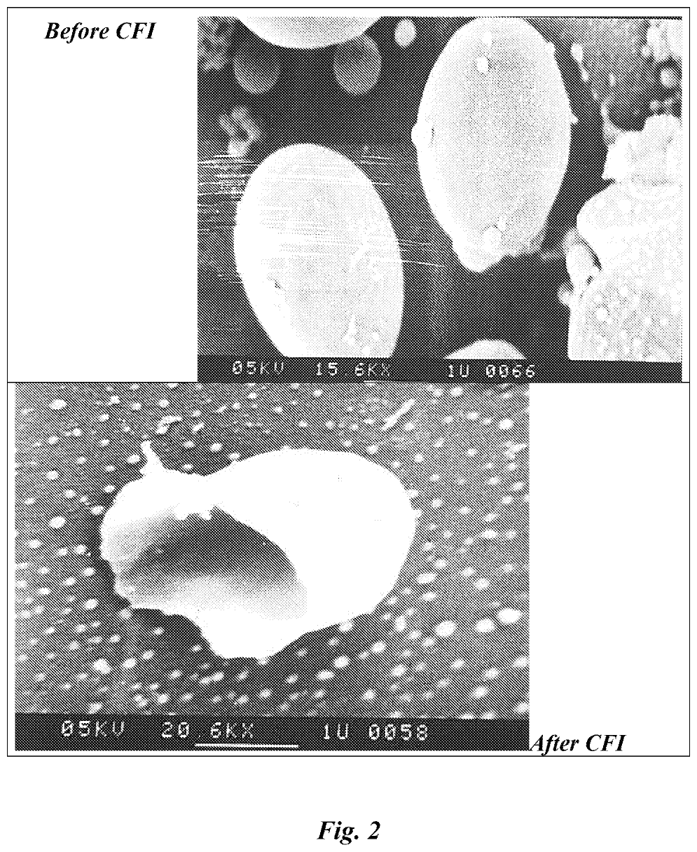

CFI has the capability to physically disrupt viral particles as shown by TEM stains of bacteriophage virus (D-6 before and after CFI treatment in FIG. 1, and by SEM photomicrographs of yeast before and after CFI treatment in FIG. 2 illustrating its ability to inactivate enveloped viruses and a variety of other tough microorganisms. Also, like the SD technique developed by the New York Blood Center, CFI inactivates enveloped viruses by a lipid solubilization mechanism, dissolving away the protective lipid coat. The CFI process is compared to select commercially available virus inactivation processes in Table 1.

TABLE 1
Summary of Select Competitive Pathogen (Virus) Inactivation & Clearance Technologies

| Method/Company | Strengths | Weaknesses |
|---|---|---|
| CFI ™ | Effective against enveloped & non-enveloped | New technique requiring industry |
| Aphios | viruses | acceptance |
| Corporation | Applicable to units and pools of plasma | |
| | Near ambient temperatures; short processing | |
| | times | |
| | Gentle process conditions protect biological | |
| | activity | |
| | No removal of chemical additives required | |
| | Scalable with low operating costs | |
| Ultraviolet Light | Effective against enveloped and some non- | Process is not easily scalable |
| Activated Nucleic | enveloped viruses in platelet and plasma units | Not applicable to pools of plasma |
| Acid Modification | Able to be used at small blood processing | HAV, HEV, B19, and Polio Virus |
| Cerus Corporation | establishments | resistant to this inactivation process |
| | | Requires removal of a potentially |
| | | harmful chemical additive |
| Solvent/Detergent | Effective against enveloped viruses in pooled | Not directly effective against non- |
| Treated Pooled | plasma | enveloped viruses |
| Plasma | Able to be produced at a large scale | Requires removal of chemical additives |
| Octapharma | Widely accepted method | Loss of biological activity |
| | | Not applicable to units of human plasma |
| Nanofiltration | Effective against enveloped & non-enveloped | Passive process |
| Pall, Millipore, | viruses | Nonspecific removal of proteins |
| Asahi | Effective for smaller (<180,000 MW) proteins | Removes large proteins |
| | | Not applicable to units of human plasma |

Three fundamental steps are required for CFI pathogen clearance of protein-rich solutions containing viruses. SFS is first added to the product, which is then brought to the appropriate pressure and temperature conditions. Next, the aqueous sample is mixed with the SFS. Finally, the sample is decompressed to ambient pressure. The mixing step is an area of importance in the design and engineering of continuous flow CFI equipment, since most SFS and proteinaceous solutions are relatively immiscible with each other. Mixing will affect the efficiency with which virus particles are contacted and saturated with the SFS and their subsequent inactivation. Efficient mixing will also reduce processing time, improve manufacturing throughput and significantly reduce overall manufacturing costs.

Viral inactivation time can be significantly reduced and protein loss minimized by diffusing the SuperFluids™ into laminar, small-diameter aqueous droplets or streams. This discovery was made by modeling the mass transport phenomena that occurs between an SFS phase and a laminar flow protein-rich liquid phase. The inventor hypothesized that the disruption mechanism involved diffusion of the SFS from the suspending aqueous medium into the virus particle (virion) and vice-versa. If the pressure in the surrounding medium is reduced rapidly enough, fluids that had previously diffused into the virions do not have sufficient time to diffuse out again. The expansion of these fluids into gases within the virions will disrupt the viral structure. A model for this process would account for the diffusion of the SFS out of the virion in response to the time-varying boundary condition of SFS in the media surrounding the virus. This mechanism was modeled using Fick's Law of Diffusion through a series of spherical shells and solved the time-varying boundary condition for spherical coordinates by finite element analysis. Modeling of the explosive decompression mechanism gave guidance to operating pressures, pressure drop and rate of pressure drop.

A two-stage CFI device design is shown in FIG. 7. The process flow is as follows. First, liquefied gas from a liquefied gas cylinder is filtered and cooled prior to being introduced to a high pressure syringe pump. The pressurized output SuperFluids™ is optionally mixed with a cosolvent pressurized by a cosolvent pump. The selectivity of nonpolar near-critical or supercritical fluid solvents can be further enhanced by the use of small concentrations of polar cosolvents such as ethanol, methanol or acetone. Supercritical fluids, critical or near-critical solvents with or without cosolvents are jointly referred to as SuperFluids™ (SFS).

The output pressure is controlled with a backpressure regulator. The final SuperFluids™ mixture is then heated as necessary before being introduced to a pressure vessel input manifold. This manifold leads to individual modular SuperFluids™ vessels. Bypass valves are in place for operations outside of the CFI operating conditions like cleaning. A filter is in place on the output to capture any debris materials. The pressure of the output SuperFluids™ is controlled by a high pressure normally closed pressure control solenoid valve. This valve can be bypassed in case of electrical failure. An expansion tank is in place to help dampen the rapid expulsion of gas during decompression. The input and output are both isolated. The pressure of the decompression chamber is controlled by a backpressure regulator. A HEPA filter is in place to ensure that only clean gas exit the system. A muffler follows the HEPA filter to dampen the sound of the exhaust in the lab. Both chambers have drain valves for system cleaning.

The effect of different ratios of SuperFluids™ CO₂ and N₂O on pH and coagulation factors in human plasma was evaluated on an extant two-stage CFI unit to select the best composition of SFS for pathogen inactivation. Plasma was pumped through a dual barometric chamber where the first chamber was pressurized to 3,000 psig and the second chamber was pressurized to 2,000 psig.

Studies were first carried out to determine the effects of SFS N₂O and CO₂ on the pH of the plasma. Treatment of pooled human plasma with SFS N₂O and 27 ppm CO₂ raised the pH from 7.9 to 8.14 while SFS CO₂ alone lowered the pH to 7.16. These results suggested that an increase in the concentration of CO₂ in SFS N₂O was needed in order to prevent an increase in pH while maintaining a pH close to 7.9. Normal pooled human plasma had a pH of 7.87 before being exposed to the SFS.

Additional studies were conducted to assess the effect of SFS treatment on the pH of plasma obtained from Innovative Research, Novi, Michigan. It was found that a 97.5:2.5 SFS mixture of N₂O:CO₂ resulted in the least change in pH and had the least impact on coagulation factors. The pH of the control plasma was relatively high and could be the result of the plasma age. Similar experiments were conducted with human plasma obtained from the Rhode Island Blood Center. This plasma had a pH of 7.4 and the best SFS mixture conditions for this plasma were also 97.5:2.5::N₂O:CO₂.

Process Validation Using Model System/Biologics: As an intermediate layer of process and functional optimization of the bench-top CFI devise, we used selected proteins to again assess and validate the effect of CFI technology on protein physiologic integrity before applying the technology to human blood plasma studies. This was conducted prior an assessment of CFI on units of plasma biologics, adopting the methods and conditions established under preliminary studies, but using the innovative bench-top CFI platforms. This step is designed to create optimal and repeatable conditions using individual commercial proteins in buffered conditions, that particularly focus on assessing TT, PT (INR), Fib and APTT and other variables tested earlier in Tables 8, 9, 10, 11 and 12, including assessment of effect of CFI treatment on: (i) matrix pH; and (ii) synthetic or purified protein aggregation. These studies inform experimental design and approaches for plasma and virus inactivation studies in units of human plasma using the benchtop device.

The detailed description set forth above is provided to aid those skilled in the art in practicing the present invention. However, the invention described and claimed herein is not limited in scope by the specific embodiments herein disclosed. The embodiments are intended as illustrations of several aspects of the invention. Any equivalent embodiments are intended to be within the scope of this invention. Various modifications of the invention in addition to those shown and described herein will become apparent to those skilled in the art from the foregoing description which do not depart from the spirit or scope of the present inventive discovery. Such modifications are also intended to fall within the scope of the appended claims.

### EXAMPLES

### Example 1: SuperFluids™ CFI Laminar Flow Viral Inactivation of an Enveloped Virus and Incremental Inactivation by Adding Stages

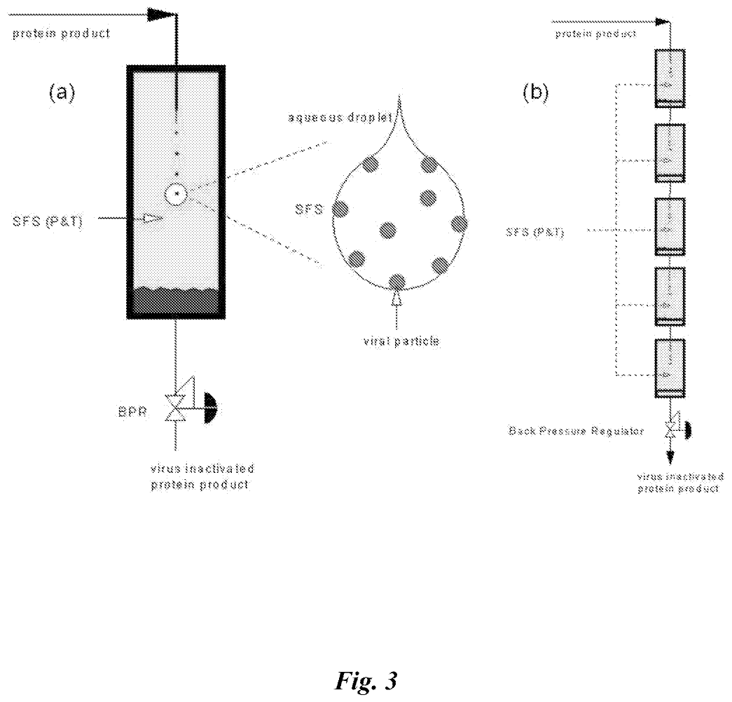

Testing in a single-stage laminar flow CFI device demonstrated that inactivation levels can be significantly increased and residence times can be significantly reduced by contacting the SuperFluids™ with small-diameter aqueous droplets or streams (Castor et al., 1999, 2000, 2001 and 2002). The basic concept is to inject an aqueous droplet or stream into an isobaric mixing chamber containing the SFS as shown in FIG. 3a.

Since the SFS is usually immiscible with the aqueous phase, virions on the surface of the droplet are more likely to be contacted and saturated with SFS than virions in the interior of the droplet. As the droplet size decreases, the probability of having more virions on the surface and the level of inactivation increases. The time required to approach the equilibrium concentration of SFS by diffusion into the interior of an aqueous droplet can be tailored by choosing the injector inner diameter, length of the mixing or drop section and flow rate. Volume throughput can be scaled by increasing the cross-sectional area of the isobaric chamber. More significantly, inactivation levels can be increased by adding stages, as shown in FIG. 3b. In going from one stage to another, the surfaces of the droplets are reformed stochastically, presenting a different spectrum of droplets and virions on their surfaces to be contacted and saturated by the SFS in the isobaric chamber. Theoretically, the level of inactivation can be thus increased by the following equation:

$$Total Logs Inactivation ≃ Logs Inactivation Per Stage \times Number of Stages + Logs Inactivation via BPR$$

. . . where BPR is the backpressure regulator that controls the final pressure reduction step (after the last stage) to atmospheric pressure

This approach confers several advantages: (1) shear forces are minimized, reducing possible damage to proteins; (2) contact of the aqueous stream with the walls of the mixer can be minimized, reducing possible protein loss; and (3) mixing geometry is simple and scalable. Volume throughput can be scaled by increasing the cross-sectional area of the isobaric mixing chamber as in chromatographic column scale-up; inactivation can be increased by adding stages as is done for improving separation efficiency in a distillation column (FIG. 3b).

In a typical experiment, a viral-loaded solution is injected into an isobaric chamber containing SuperFluids™ under pre-specified conditions of flowrate, temperature and pressure. The residence time of a droplet in a single stage CFI injection unit is less than 20 seconds. Treated samples are collected in bulk at the end of a completed run or at specified times during the run. Control and treated materials are analyzed for virus infectivity as well as protein content and integrity. Several tests (Table 2) were performed with murine-C retrovirus (MuLV) in fetal bovine serum (FBS) with N₂O at sub-optimal conditions of 139 bars and 22° C. MuLV, an enveloped virus, which has an outer diameter of approximately 100 nanometers (nm), is often used as a surrogate for human immunodeficiency virus (HIV). CFI-286 was performed by directly passing the pressurized FBS solution containing MuLV through the backpressure regulator (BPR) without being contacted with SuperFluids™.

TABLE 2
Inactivation of MuLV in FBS in SuperFluids ™ CFI Laminar
Flow Units [N₂O at 139 bars and 22° C.]

| Parameters | CFI-286 | CFI-380 | CFI-381 | CFI-464 |
|---|---|---|---|---|
| No. of Stages | 0 | 1 | 1 | 2 |
| Time (min.) | <1 | <1 | <1 | <1 |
| Titer Control | 1 × 104.0 | 1 × 106.0 | 1 × 103.0 | 1 × 105.5 |
| Titer After | 1 × 103.0 | 1 × 103.7 | 1 × 101.0 | Not Detected |
| −log10 Reduction | 1.0 | 2.3 | 2.0 | >5.5 |

The zero (0) stage experiment in CFI-286, no isobaric chamber, resulted in about 1 log of inactivation of MuLV. The single stage experiments in CFI-380 and CFI-381 (duplicate runs) inactivated 2 or more logs of MuLV in a residence time less than 20 seconds. The two-stage unit in CFI-464 inactivated more than 5.5 logs of MuLV in less than one minute. The data in Table 2 indicates that the level of inactivation by SuperFluids™ CFI can be increased by adding stages for enveloped viruses.

### Example 2: SuperFluids™ CFI Laminar Flow Viral Inactivation of a Nonenveloped Virus and Incremental Inactivation by Adding Stages

Several experiments (similar to that in Table 2) were conducted with Encephalomyocarditis (EMC), a tough, prototypical non-enveloped or protein-encased virus at sub-optimal conditions to demonstrate the scalability of the CFI technology for non-enveloped viruses. EMC, a member of the Picornaviridae family, is a positive-strand RNA virus that is often used as a surrogate for the hepatitis A virus (HAV). The data listed in Table 3 indicate that over four logs of inactivation (4.9 and 4.2 logs) were obtained with EMC in the two-stage CFI unit. In the single-stage unit (CFI-882 and CFI-883), 3.6 and 3.5 logs were obtained. Thus, the second stage appears to add an average of one log of inactivation. Interestingly, the optimum inactivation pressure for EMC was about 140 bars (˜2,000 psig) lower for Freon-22 at 50° C.

TABLE 3
CFI of EMC in FBS in Single-Stage and Two-Stage CFI Laminar
Flow Units [Freon-22 at 345 bars and 50° C.]

| Parameters | CFI-882 | CFI-883 | CFI-894 | CFI-895 |
|---|---|---|---|---|
| No. of Stages | 1 | 1 | 2 | 2 |
| Time (min.) | <1 | <1 | <1 | <1 |
| Titer Control | 1 × 105.7 | 1 × 105.5 | 1 × 105.5 | 1 × 105.8 |
| Titer After | 1 × 102.1 | 1 × 102.0 | 1 × 100.6 | 1 × 101.6 |
| −log10 reduction | 3.6 | 3.5 | 4.9 | 4.2 |

### Example 3: SuperFluids™ CFI Laminar Flow Viral Inactivation of Several Enveloped and Nonenveloped Viruses in Single-Stage Laminar Flow CFI Unit

With Freon-22 at 208 bars and 50° C., approximately six logs of EMC were inactivated by SFS in less than 20 seconds in a single-stage laminar flow. Under similar conditions at 208 bars and 501C, CFI was also effective with other non-enveloped viruses (Adenovirus, Poliovirus, HAV, Reovirus, and Parvovirus) and enveloped viruses (VSV, Sindbis, TGE, BDVD and HIV), while often exceeding our design criterion of >1 log of inactivation per stage (Table 4), demonstrating the general applicability of the technology to both enveloped and non-enveloped viruses.

TABLE 4
CFI of Non-enveloped and Enveloped Viruses in FBS in a Single-
Stage CFI Injection Unit with Freon-22 at 208 bars and 50° C.

| | | | Size | | −log₁₀ |
|---|---|---|---|---|---|
| CFI No. | Virus | Family | (nm) | Capsid | Kill |
| 887 | EMC | Picornaviridae | 20-30 | Nonenveloped | 5.9 |
| 551 | EMC | Picornaviridae | 20-30 | Nonenveloped | 5.4 |
| 914 | EMC | Picornaviridae | 20-30 | Nonenveloped | >5.7** |
| 915 | EMC | Picornaviridae | 20-30 | Nonenveloped | >5.6** |
| 916 | Adeno | Adenoviridae | 70-90 | Nonenveloped | >5.3** |
| 917 | Adeno | Adenoviridae | 70-90 | Nonenveloped | >5.1** |
| 918 | Polio | Picornaviridae | 18-26 | Nonenveloped | 4.1 |
| 919 | Polio | Picornaviridae | 18-26 | Nonenveloped | 4.1 |
| 908 | HAV | Picornaviridae | 24-30 | Nonenveloped | 1.3 |
| 909 | HAV | Picornaviridae | 24-30 | Nonenveloped | 1 |
| 898 | Reo | Reoviridae | 65-75 | Nonenveloped | 0.9* |
| 889 | Reo | Reoviridae | 65-75 | Nonenveloped | 1.0* |
| 1013 | Parvo | Picornaviridae | 18-26 | Nonenveloped | 1.5 |
| 1014 | Parvo | Picornaviridae | 18-26 | Nonenveloped | 1.6 |
| 904 | VSY | Rhabdoviridae | 60-180 | Enveloped | >6.5** |
| 905 | VSV | Rhabdoviridae | 60-180 | Enveloped | >6.5** |
| 906 | Sindbis | Togaviridae | 60-70 | Enveloped | >6.5** |
| 907 | Sindbis | Togaviridae | 60-70 | Enveloped | >6.5** |
| 902 | TGE | Coronaviridae | 80-130 | Enveloped | >2.5** |
| 903 | TGE | Coronaviridae | 80-130 | Enveloped | >2.5** |
| 900 | BDVD | Togaviridae | 60-70 | Enveloped | 2.3 |
| 901 | BDVD | Togaviridae | 60-70 | Enveloped | 2.3 |
| 464 | MuLV | Retroviridae | 80-100 | Enveloped | >5** |
| VAC-6 | HIV-1 | Retroviridae | 100-120 | Enveloped | >5.7** |
| VAC-6^{†} | HIV-1 | Retroviridae | 100-120 | Enveloped | >5.3** |

*3.7 logs of inactivation have been obtained in a two-stage CFI injection unit.
^{†}With SuperFluid Fr-22.
**UD; Undetectable virus, the highest of lower limit of detection used for calculation

### Example 4: SuperFluids™ CFI Laminar Flow Viral Inactivation of HIV in a Single-Stage Laminar Flow CFI Unit

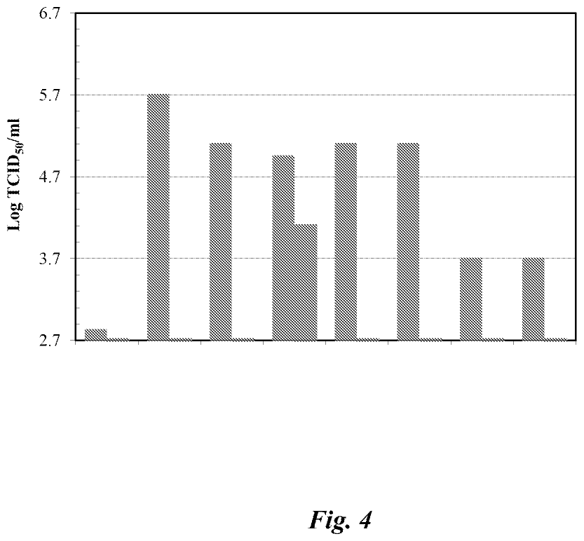

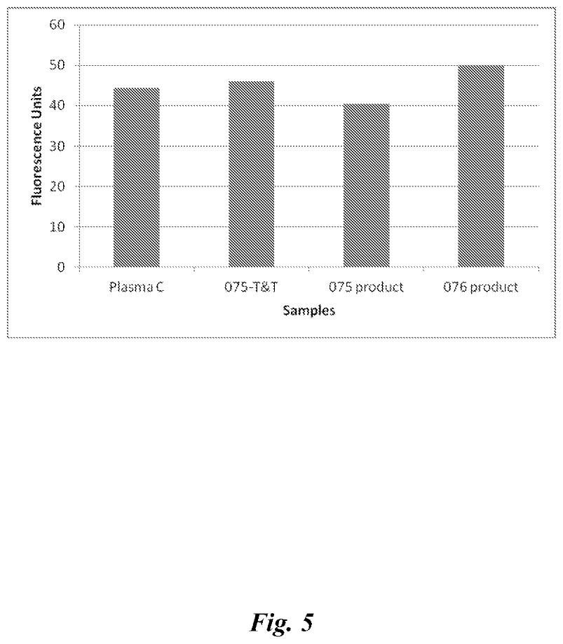

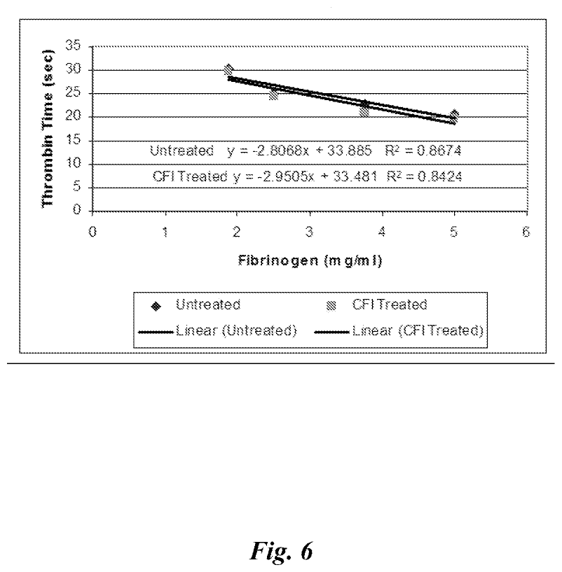

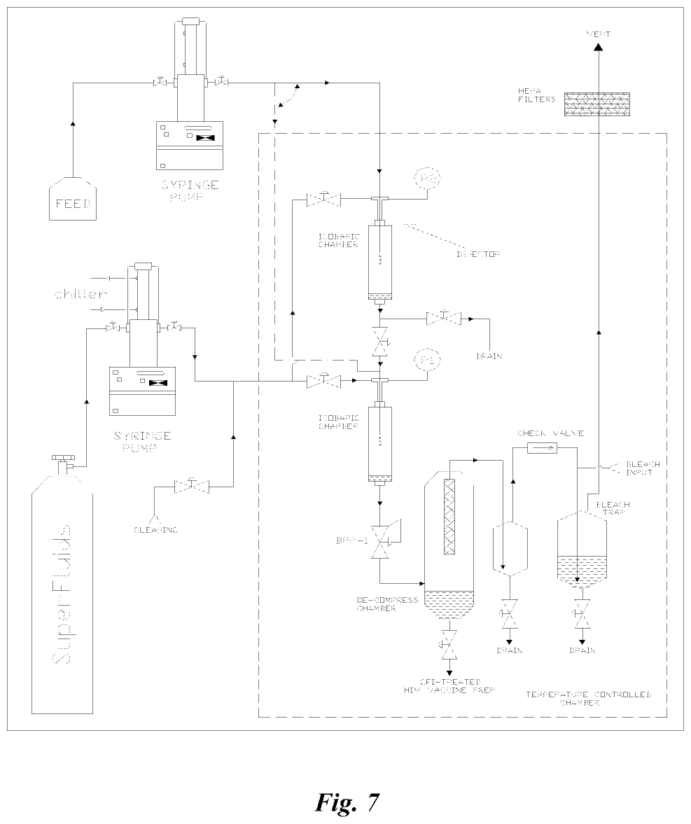

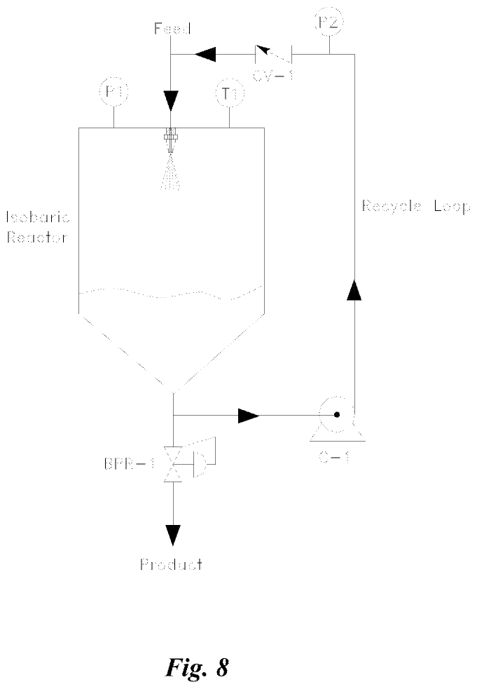

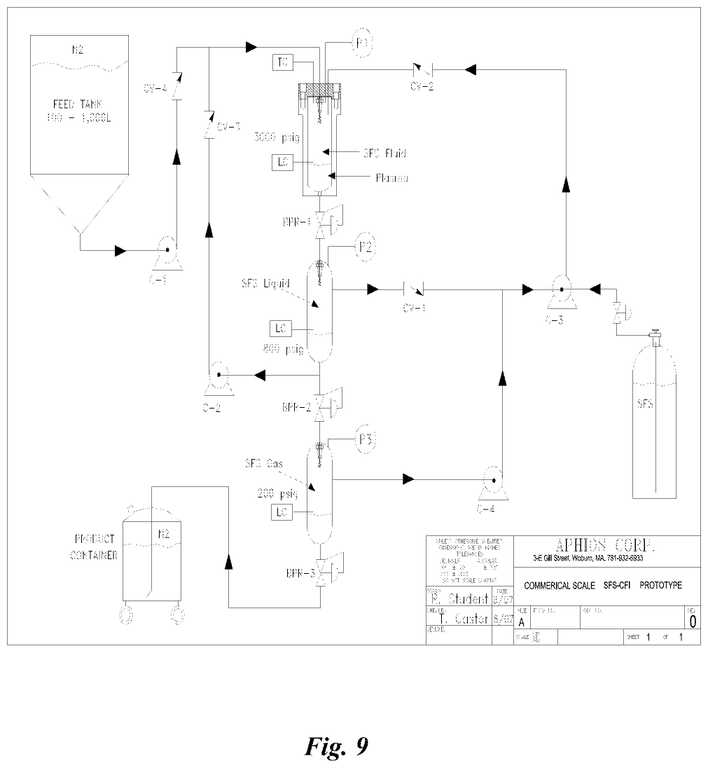

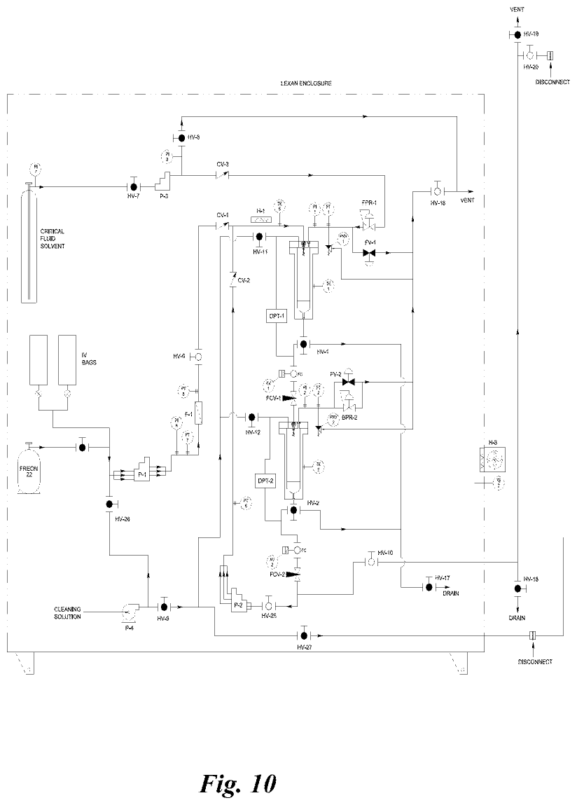

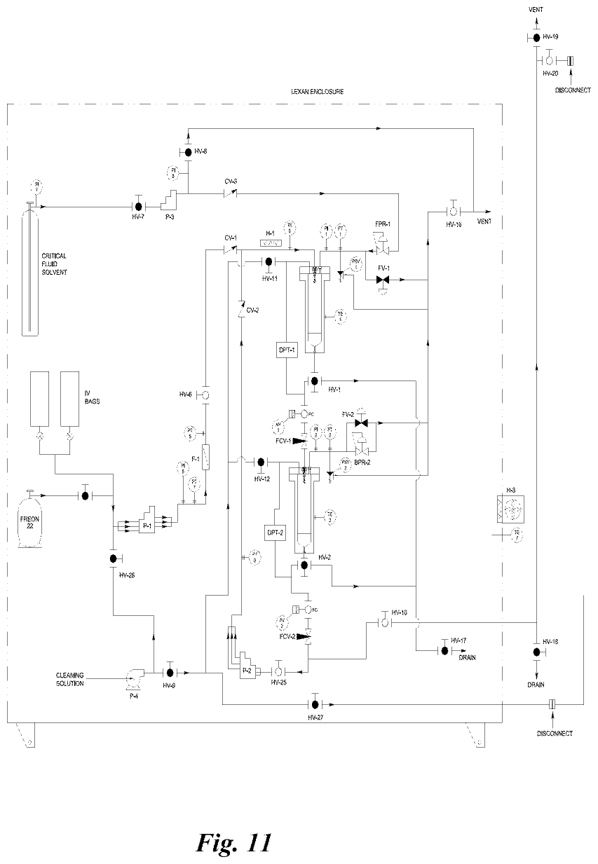

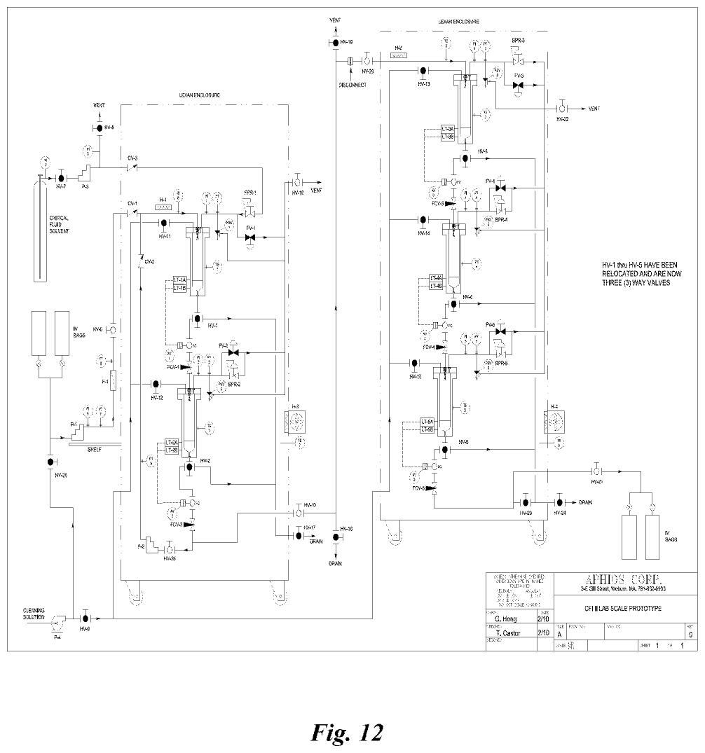

Inactivation experiments were performed in a single stage CFI unit to explore the use of different SFS, at 208 bars and 22° C., for treatment of an HIV laboratory strain, HIV_{Δtat-rev}, which lacks the accessory genes tat and rev. Seven SFS were tested for their ability to inactivate HIV, including N₂O, N2, propane, Fr-22, and a N₂O/CO₂ mixture [limited data shown in FIG. 4]. All were utilized at room temperature, which during comparable exposure times had no effect on HIV stability. A single-stage laminar flow rate of 4 mL/min was used at 208 bars and 22° C.

With the exception of propane, infectious virus could not be detected in the CFI-treated samples as compared to untreated controls, indicating complete inactivation. The maximum reportable log inactivation (>5.7 logs) was obtained when NO₂O/CO₂ (N₂O with trace amounts of CO₂) was used with the highest titer HIV N₂O/CO₂ was chosen for use because we had previously shown little or no toxicity on a variety of cell lines, and this mixture is relatively inexpensive. In all these and subsequent experiments similar amounts of p24 were observed in CFI-treated and untreated samples.

### Example 5: SuperFluids™ CFI Laminar Flow Viral Inactivation of Parvovirus B19 in a Single-Stage Laminar Flow CFI Unit

In a research collaboration with the National Institute of Biological Standards and Control (NIBSC), London, England, the inventor demonstrated that CFI can inactivate at least logs of human Parvovirus B19 in human serum in a two-stage CFI unit (Table 5), rendering original virus undetectable. Samples of parvovirus B19-spiked in human plasma free of B19 antibodies were provided by NIBSC, CFI-treated by Aphios and blinded samples were shipped back to NIBSC for testing. The samples were CFI-treated with three supercritical fluids (Freon-22, Freon-23 and N₂O/CO₂) at either 25° C. or 50° C. In NIBSC-01, with SuperFluids™ Freon-22 at 208 bars and 50° C. in a two-stage laminar flow CFI unit, there was approximately a 2 log₁₀ change in infectivity titer compared with the untreated sample.

TABLE 5
B19 Infectivity Assay of CFI-Treated Samples and Controls

| | | | Infectious Units per mL at; | | |
|---|---|---|---|---|---|
| Expt. | | T | | Time & | |
| No | SuperFluids ™ | (° C.) | Before (4° C.) | Temperature (t&T) | CFI-Treated Samples |
| 01 | Freon-22 | 50 | 1 × 10⁵ | 5 × 10^{4.5} | 5 × 10³ |
| 02 | Freon-22 | 25 | 3 × 10⁵ | 7 × 10⁴ | 2 × 10⁵* |
| 03 | Freon-23 | 50 | 2 × 10⁴ | 1.7 × 10^{4.5} | NS |
| 04 | Freon-23 | 25 | 3 × 10^{4.5} | 1 × 10⁶ | 1.7 × 10⁶* |
| 05 | N₂O/CO₂ | 50 | 1 × 10⁴ | 5 × 10^{4.5} | No detectable |
| | | | | | infectious particles* |
| 06 | N₂O/CO₂ | 25 | 2 × 10^{5.5} | 2 × 10⁶ | 1.3 × 10⁶* |

The “time and temperature” control sample had a similar infectious titer to the untreated sample indicating that the loss of infectivity was due to the treatment rather than incubation of the sample at 50° C. In NIBSC-05, SuperFluids™ CFI inactivated more than 4 log₁₀ of parvovirus B19 spiked into plasma by N₂O/CO₂ was inactivated at 208 bars and 50° C. in a two-stage laminar flow CFI unit with no detectable infectious particles remaining. The inactivation levels appear to be sensitive to SFS type with higher levels attained with N₂O/CO₂ versus Freon-22 and Freon-23, and temperature with higher levels attained at 50° C. versus 25° C. The absolute effect of temperature by itself was negligible and accounted for by time and temperature controls.

### Example 6: Optimum SFS Mixture of CFI for Human Plasma

Using human plasma at physiological pH, the effect of treatment with different ratios of SFS CO₂ and N₂O on pH and coagulation factors was further evaluated to select the best composition of SFS for pathogen inactivation. Plasma was pumped through a dual barometric chamber where the first chamber was pressurized to 3,000 psig (208 bars) and the second chamber was pressurized to 2,000 psig (138 bars).

At least 5 variations of SuperFluids were tested including; (1) 100%::CO₂. (2) 90:10::N₂O:CO₂, (3) 95:5::N₂O:CO₂, (4) 97.5:2.5::N₂O:CO₂ and (5) 100%::N₂O. Table 6 presents the results of the study with 97.5:2.5 SFS mixture of N₂O:CO₂ which resulted in the least change in pH and had the least impact on coagulation factors. The control pH of the plasma (from Innovative Research, MI) was relatively high and could be the result of the plasma age. Repeat experiments were conducted with human plasma obtained from the Rhode Island Blood Center (RIBC). This plasma had a pH of 7.3 and the best SFS mixture conditions were also 97.5:2.5::N₂O:CO₂ for the repeated experiments.

TABLE 6
Effect of SuperFluids ™ on the Coagulation
Factors of Human Pooled Plasma

| | pH | Factor VIII | TT | APTT | PT |
|---|---|---|---|---|---|
| Control | 7.96 | 45.5%, 59.1 s | 13.3 s | 32.5 s | 14.1 s |
| SFS treated | 7.78 | 33.5%, 62.3 s | 16.1 s | 37.1 s | 17.3 s |
| % Change | −2.26 | −26.4%, 5.4 s | 21.05 | 14.15 | 22.70 |

### Example 7: SuperFluids™ CFI Virus Inactivation Studies with an Optimized SFS Mixture Consisting of N₂O:CO₂::97.5%:2.5% at Different Temperatures in Single- and Two-Stage CFI Units

CFI virus inactivation studies with an optimized SFS mixture consisting of N₂O:CO₂::97.5%:2.5% at different temperatures in single- and two-stage CFI units are summarized in Table 7. Bovine viral diarrhea virus (BVDV), the tough prototypical model for the enveloped virus Hepatitis C, was undetectable after single-stage CFI treatment at 207 bars and 50° C. (CFI-I-024)—representing >4.3 logs of inactivation.

TABLE 7
CFI Virus Inactivation Studies with SFS N₂O:CO₂::97.5%:2.5%

| CFI | No. of | | | Temp | Pressure | t&T | CFI | |
|---|---|---|---|---|---|---|---|---|
| No. | Stages | Matrix | Virus | (0° C.) | (bars) | titer | Titer | −log₁₀Kill |
| 024 | 1 | Plasma | BVDV | 50 | 208 | 5.98 | UD | >4.34* |
| 037 | 1-recycle | Plasma | BVDV | 40 | 208 | 5.35 | 2.4 | 2.87 |
| 025 | 1-recycle | Plasma | BVDV | 50 | 208 | 5.73 | 5.1 | 0.63 |
| 030 | 1 | FBS | HAdV | 50 | 208 | 4.73 | UD | >3.09* |
| 032 | 1 | FBS | HAdV | 50 | 208 | 7.98 | UD | >6.34* |
| 033 | 1-recycle | FBS | HAdV | 50 | 208 | 7.98 | UD | >6.34* |
| 034 | 1 | FBS | HAdV | 40 | 208 | 8.48 | 5.23 | 3.25 |
| 035 | 1-recycle | FBS | HAdV | 40 | 208 | 8.23 | 4.1 | 4.13 |
| 041 | 1 | FBS | EMCV | 40 | 208 | 7.35 | 5.1 | 2.25 |

*UD; Undetectable virus, the highest of lower limit of detection used for calculation

Since PPV (porcine parvovirus) is not a good prototype for human parvovirus B19, the inventor elected to evaluate the impact of the optimum SFS mixture on the non-enveloped human Adenovirus (HAdV) Type 2 virus. These studies were performed using FBS instead of human plasma since the latter neutralized the virus due to the presence of antibodies to adenovirus.

The inventor obtained complete CFI inactivation of HAdV by SFS N₂O:CO₂::97.5%:2.5% at 207 bars and 50° C. using both a single-stage CFI unit and single-stage without recycle (CFI-I-032 and 033, respectively). At 40° C., CFI resulted in 3.25 logs inactivation in a single-stage CFI unit and 4.13 logs in a single-stage CFI unit with recycle (CFI-I-034 and 035, respectively). Mouse Encephalomyocarditis virus (EMCV), a picorna virus considered very resistant to inactivation, was also inactivated 2.25 logs by CFI at 208 bars and 40° C. (CFI-I-041) in a single stage CFI unit.

### Example 8: SFS Treatment of Serum Did not Adversely Affect Cell Function

The inventor tested the ability of SuperFluids™ CFI treated fetal bovine serum, human plasma proteins such as Factor VIII and immunoglobulins, sensitive natural enzymes such as alkaline phosphatase and α₁-protease inhibitor and recombinant proteins such as biosynthetic insulin to retain biochemical characteristics and biological activity. Several aliquots of a commercial fetal calf serum (FCS) were treated with N₂O/CO₂ at 2,000 psig and 22° C. and compared with untreated controls by SMAC analysis and by examining the growth characteristics of several cell lines (Table 8).

TABLE 8
Effect of SuperFluids ™ on Doubling Rate and Plating
and Cloning Efficiencies of Various Cell Lines

| | Cell type and Density (cells/ml) | | | | | |
|---|---|---|---|---|---|---|
| Time | HeLa | | A549 | | 3T6 | |
| (Days) | Control | CFI-treated | Control | CFI-treated | Control | CFI-treated |
| 1 | 300,000 | 100,000 | 500,000 | 400,000 | 400,000 | 200,000 |
| 2 | 120,000 | 120,000 | 700,000 | 700,000 | 700,000 | 700,000 |
| 3 | 1,300,000 | 990,000 | 1,200,000 | 1,200,000 | 1,000,000 | 1,300,000 |
| 4 | 1,100,000 | 1,100,000 | 1,400,000 | 1,600,000 | 8,000,000 | 8,000,000 |
| 6 | 5,100,000 | 4,900,000 | 9,000,000 | 7,000,000 | 10,000,000 | 10,000,000 |
| 8 | 10,000,000 | 10,000,000 | 10,000,000 | 10,000,000 | 10,000,000 | 10,000,000 |

CFI treatment had no effect on total protein, lactic dehydrogenase or alkaline phosphatase levels, with treated tests being within 90% of untreated FCS (data not shown). The CFI-treated FCS was used to maintain the cell lines in culture after which cytotoxicity, doubling rate, plating efficiency (time to confluency), and cloning efficiency were determined. CFI-treated FCS was within 80% to 100% of untreated FCS in these tests (Table 8). Thus, CFI treatment had no or insignificant effect on the serum proteins, enzymes, and cytokines needed for cell function. These results were confirmed independently by BioWhittaker, Walkerville, MD (data not shown).

### Example 9: Effect of CFI Treatment on pH of Plasma

The results of the effect of CFI treatment on pH are shown in Table 9 for a 97.5%:2.5% mixture of N₂O:CO₂. The pH increased by about 0.4 units in the two flow samples presumably due to the loss of carbon dioxide. It is worth noting that this increase also occurs for plasma during routine storage if there is a large dead air space available. The pH of the untreated plasma was 7.3 when tested as soon as it was drawn out of the plasma bag, but increased to 7.65 after a few hours storage in a 50 mL conical tube.

TABLE 9
Effect of SFS-CFI
97.5%:2.5%::N₂O:CO₂
Treatment on Plasma pH

| Sample | pH |
|---|---|
| Untreated | 7.65 |
| 5 mL/min | 8.05 |
| 20 mL/min | 8.09 |
| Pump Residue | 7.64 |

### Example 10: Effect of CFI Treatment on Plasma Clotting Characteristics

The effect of CFI treatment on plasma clotting characteristics as measured in ACL 3000 are shown in Tables 10 and 11. Based on the nominal acceptance criteria of percent change in activity of at least 20% for the treated compared to untreated or normal samples, the SFS treated samples showed acceptable values for all criteria tested, and was within normal INR.

TABLE 10
Effect of CFI 97.5%:2.5%::N₂O:CO₂ Treatment
on Plasma Characteristics - ACL Results

| | | | % | | % | | % | | % |
|---|---|---|---|---|---|---|---|---|---|
| Sample | PT* | INR* | change | TT | change | APTT | change | Fib | change |
| Untreated | 12.8 | 1.02 | — | 16.3 | — | 29.6 | — | 271 | — |
| SFS Treated | 12.8 | 1.02 | 0 | 17.9 | 9.82 | 32.3 | 9.12 | 250 | −7.75 |

Note:
PT, TT and APTT are shown in seconds and Fib is shown in mg/dL (deciliter = 100 mL).
Expected calibration range: PT, 10 s-13 s; PT normal = 12.6 s (IL Lot#N0799679). APTT, 25-34 s.
*ISI for PT to INR conversion = 1.63. INR, International Normalized Ratio.

TABLE 11
Impact of CFI on Human Plasma Proteins

| | Conditions | | | Parameters | | | | | |
|---|---|---|---|---|---|---|---|---|---|
| CFI-I | N₂O:CO₂ | T(° C.) | P (bars) | PT | Fib | TT | APTT | Factor VIII | pH |
| 61 | 97.5:2.5 | 40 | 208 | 103% | 90% | 103% | 107% | ND | 112% |
| 65 | 97.5:2.5 | 40 | 208 | 106% | 81% | 112% | 115% | 108% | 108% |
| 67 | 97.5:2.5 | 37 | 208 | 102% | 91% | 108% | 110% | 105% | 105% |

### Example 11: Effects of CFI on Human Plasma Proteins

The effect of CFI on human plasma over the planned operational range of 97.5:2.5::N₂O:CO₂ at 208 bars and 40° C. was evaluated (Table 11). An additional data set at 208 bars and 37° C. is included to represent a potential operation at body temperature (CFI-I-67). At test conditions, the normalized values of the results for ACL assays and pH showed that pro-thrombin time (PT) varied from 102-106%, fibrinogen from 81 to 91%, thromboplastin time (TT) from 103 to 112%, activated partial thromboplastin time (APTT) from 110 to 115%, Factor VIII from 99-108%, and pH from 105 to 112%. These data showed between 10-20% loss in fibrinogen, which appears to be an outlier in the pilot studies as the integrity of the other four proteins studied were preserved. Rigorous evaluations of all these factors are further envisaged in this application.

### Example 12: Effect of CFI on Plasma Protein Aggregation

Proteostat protein aggregation assay (Enzo Life Sciences, Farmingdale, NY) was performed to determine the effect of CFI treatment on protein aggregation of plasma treated by CFI at different pressures and temperatures —N₂O:CO₂::97.5:2.5 at 104 bars and 30° C. [CFI-I-075] and N₂O:CO₂::97.5:2.5 at 208 bars and 40° C. [CFI-I-076]. These assays were performed as per the manufacturer's recommendations for CFI-I-075 and 076. The assay was run in duplicate for the samples diluted 1:10. The results (FIG. 5) showed about a ±10% variance.

In addition, denaturing-reducing and native gels were run for CFI-I-075 and −076 samples to determine any losses of protein bands or shifts in molecular weights by CFI treatment. In these experiments, products collected immediately at the end of the run (t=0) as well as the products collected after 20 minutes (t=20), i.e. material accumulated in the depressurization chamber after the run) were analyzed. There was no observed loss of bands or change in molecular weights for any of the samples.

### Example 13: Effect of CFI on Fibrinogen

The effect of CFI N₂O at 152 bars and 22° C. for 1 hour on fibrinogen is shown in FIG. 6, demonstrating no difference in thrombin time versus control over a range of fibrinogen concentrations tested.

### Example 14: Effect of CFI on Hyperimmunoglobulin

The effects of CFI N₂O on a hyperimmunoglobulin at different temperatures (22 to 40° C.) and pressures (0 to 278 bars) are listed in Table 12 and compared to controls at atmospheric pressure showing little or no change in physical and potency parameters tested.

TABLE 12
Effect of CFI N2O at different pressures
and temperatures on a Hyperimmunoglobulin

| NO₂ | HPLC- | Anti- | Protein | ELISA |
|---|---|---|---|---|
| bars/° C. | SEC (%) | Complementary | (mg/ml) | MEP Abs |
| 0/22 | 94.7 | >1.74 | 18.14 | 379.5 |
| 278/22 | 95.2 | >1.74 | 17.39 | 370.8 |
| 0/29 | 101.4 | >1.83 | 18.27 | 349.7 |
| 208/29 | 92.7 | >1.77 | 17.65 | 313.8 |
| 0/40 | 104.3 | >1.81 | 18.00 | 351.4 |
| 278/40 | 99.7 | >1.78 | 17.84 | 385.4 |

### Example 15: CFI Inactivation Using a Laminar Flow Unit with Recycle

In order to increase the inactivation level in the isobaric chamber to a minimum of 3 logs for difficult-to-inactivate viruses, we can also recycle some of the protein-rich stream back into the inlet stream, as shown in FIG. 8. This recreates new surfaces so that stochastically, the probability of virions being on the surface of a droplet will increase, and the degree of virions saturated by the SFS will increase and levels of inactivation should increase. This concept can potentially replace the need for multiple stages that appear to work by the same mechanism. The variables are the recycle rate compared to feed and product recovery rates, which should be maintained the same, after start-up transients, during steady-state operation. By varying the ratio of the recycle rate to feed rate from 1 to 10, the anticipated results will be between 3 and 5.

### Example 16: Commercial-Scale CFI Inactivation Using a Laminar Flow Unit with Recycle

FIG. 9 illustrates a commercial-scale SFS-CFI prototype for processing up to 1000 liters of human plasma per day under cGMP conditions with a turndown ratio of 10:1. The best available methods were incorporated for controlling the process, and performing cleaning steps essential for validation of the unit. Our design criteria were to reduce the complexity so that it is more like a CEP (Chemical Engineering Process) operation without the added complexity of chemical reactions.

CEP fundamentals were used to stage and scale the process, fundamentals that are routinely utilized by engineers and operators in a biologics manufacturing environment. CFI has been evaluated over a flowrate of 2 to 5 Liters/8 hr day (4 to 10 mL/min). For similar type SuperFluids™ application for the extraction and purification of pharmaceuticals and nutraceuticals, we typically conduct research at flow rates of 0.5 to 2.0 mL/min and scale these results successfully to 2,000 to 5,000 mL/min in a single step. Multi-staging or second pass of solution through a chamber is practiced in many applications in the CEP industry and will not be a significant barrier to scale-up and commercialization. The potential for clogging the device is much higher on a small scale than on a large scale because of the difference in surface area to volume ratios. We have not seen clogging in the laboratory-scale prototype and thus do not expect significant clogging problems in commercial-scale CFI units.

### Example 17: Commercial-Scale Multistage CFI Inactivation Using a Laminar Flow Unit with Recycle

A multistage commercial-scale unit for processing large amounts of plasma, including plasma and SFS recycled loops, is illustrated in FIGS. 10 through 12. This system is a scaled-up version including a five-stage CFI unit design comprises five isobaric mixing chambers with continuous flow injection, capable of processing up to 1,000 liters of human plasma per day under cGMP (scale down version is 10 to 100 liters a day), with a downturn ratio of 10:1. This design incorporates best available method for controlling the process and cleaning the system of contaminants. The five-stage design was designed to operate in two modules.

In the first module, shown in FIG. 10, the plasma is re-circulated at rate of 10× the incoming stream to improve contact with the SFS. This module is a detailed design of the plasma recycle concept shown in FIG. 8. The second module of the 5-stage CFI design is a 3-stage CFI unit shown in FIG. 11. This module can be connected to the two-stage module to develop five linear stages without recycle. These two modules are combined to establish the 5-stage CFI module shown in FIG. 12.

The major design components of the five-stage CFI unit are isobaric chamber, nozzle design, liquid level controller, and programmable computer control system. Other components include temperature and pressure transducers, heaters and fans, manual and automatic valves as well as high pressure delivery and recirculation pumps. The isobaric chamber was designed based on mathematical modeling and computational fluid dynamics that encompass both the transport phenomena occurring during the SFS contact, saturation, and viral inactivation process and the droplet sizes and spray pattern generated by the nozzle and fluid streams.

What is claimed is:

1. An apparatus for inactivating viruses and other pathogen in blood plasma and other biologics, comprising:
(a) a pressure vessel for containing SuperFluids [SFS] consisting of a critical, supercritical or near critical fluid with or without small molar concentrations of cosolvents at a specified pressure and temperature;
(b) the vessel having an inlet and outlet; the inlet for introducing into the vessel droplets of the biologic, said biologic droplets containing virus and pathogen particles; the outlet including a back pressure regulator for controlling the pressure and temperature inside the vessel to maintain said SuperFluids as a critical, supercritical, or near critical fluid;
(c) wherein the biologic droplets, introduced into the pressure vessel, contact the SuperFluids which permeate and saturate the virus and pathogen particles;
(d) a process controller for controlling processing time, wherein, after a specified processing time, the SuperFluids is decompressed using the back-pressure regulator, causing rapid disruption of the virus and pathogen particles to render the virus or pathogen inactive; and
(e) a valve for removing the processed biologic from the vessel.

2. The apparatus of claim 1, configured to process 10 liters to 1,000 liters of human plasma and other biologics daily.

3. The apparatus of claim 1, configured to inactivate both enveloped and non-enveloped viruses in human plasma and other biologics.

4. The apparatus of claim 1, configured to inactivate pathogenic bacteria and parasites in human plasma and other biologics.

5. The apparatus of claim 1, further including a cosolvent pump for mixing and pressurizing cosolvents for introduction into the pressure vessel.

6. The apparatus of claim 1, further including plurality of identical pressure vessels arranged as stages in tandem, so that processing of the human plasma and other biologics proceed from one vessel to the next to increase the efficiency and levels of virus and pathogen reduction.

7. The apparatus of claim 1, further including a biologic recycling unit for recycling the biologic back into the inlet stream to cause virions to move to the surface of the droplets for saturation by the SFS to increase levels of inactivation.

8. The apparatus of claim 1, wherein the apparatus is upwardly scalable to process higher flow rates of biologics.

9. The apparatus of claim 1, configured to preserve the protein integrity and biological potency of the treated biologics during inactivation of viruses and pathogens.

10. The apparatus of claim 1, configured to operate as a continuous flow unit.

11. The apparatus of claim 1, wherein the apparatus is housed in a self-contained transportable unit.

12. A five-stage apparatus for inactivating virus and pathogens from human blood plasma and other biologics, comprising:
(a) a two-stage biologic recycle unit for recirculating biologics at the rate of 10× the incoming stream to improve contact with the SFS; and
(b) a three-stage CFI unit coupled to the output of the recycling unit for processing biologics linearly.

13. The apparatus of claim 12, further including means to couple all 5 stages linearly.

14. The apparatus of claim 12, further including a biologics recycling unit for recycling the biologics back into the inlet stream to cause virions to move to the surface of the droplets for saturation by the SFS to increase levels of inactivation.

15. The apparatus of claim 12, configured to operate as a continuous flow unit.

16. The apparatus of claim 12, wherein the unit is housed in a self-contained transportable unit.

17. A method for inactivating viruses and other pathogen in units of blood plasma and other biologics, wherein droplets of the biologic contain virus and pathogen particles, comprise the steps of:
(a) introducing droplets of biologics into an isobaric chamber containing a SuperFluids [SFS], a critical, supercritical or near critical fluid at a specified pressure and temperature; the chamber having an inlet for introducing into the vessel droplets of the biologic and the outlet including a back pressure regulator for controlling the pressure and temperature inside the chamber to maintain said SuperFluids as a critical, supercritical, or near critical fluid;
(b) contacting the biologic with the SuperFluids to permeate and saturate the virus and pathogen particles;
(c) processing the biologic droplets for a specified processing time;
(d) decompressing the SuperFluids using the back-pressure regulator, causing rapid disruption of the virus and pathogen particles to render the virus or pathogen inactive; and
(e) removing the processed biologic from the vessel.

18. The method of claim 17, wherein the process step is repeated through a series of tandem processing stages to increase the efficiency and levels of viral and pathogen reduction.

19. The method of claim 17, wherein the biologic is recycled back into the inlet stream to cause virions to move to the surface of the droplets for saturation by the SFS to increase levels of inactivation.

20. The method of claim 17, wherein the integrity of the proteins and enzymes are maintained throughout the inactivation process.
